(19) United States
(12) Patent Application Publication
(10) Pub. No.: US 2025/0152479 A1
(43) Pub. Date: May 15, 2025

(54) STABLE, LOW VISCOSITY OIL-IN-WATER EMULSIONS FOR SKIN CONDITIONING

(71) Applicant: DECIEM BEAUTY GROUP INC., Toronto (CA)

(72) Inventors: Anna Eunjin PARK, Toronto (CA); Flora Sze Wing TSE, Markham (CA); Prudvi Mohan KAKA, North York (CA)

(73) Assignee: DECIEM BEAUTY GROUP INC., Toronto, ON (CA)

(21) Appl. No.: 18/840,484

(22) PCT Filed: Feb. 22, 2022
(86) PCT No.: PCT/CA2022/050248

§ 371 Date: Aug. 21, 2024

(51) Int. Cl. A61K 8/06; A61K 8/31; A61K 8/39; A61K 8/60; A61K 8/86; A61Q 19/00

(52) U.S. Cl. CPC A61K 8/062; A61K 8/31; A61K 8/39; A61K 8/604; A61K 8/86; A61Q 19/007; A61K 2800/21; A61K 2800/52; A61K 2800/82

(57) ABSTRACT

A novel cosmetic skin care composition is provided which comprises a low viscosity oil-in-water emulsion which is stable despite having a high proportion of oil. The invented formulations are suitable for application to all skin, with several embodiments described herein being configured to be particularly beneficial for hard-to-reach areas like the scalp. A method of formulating said composition as a low viscosity stable emulsion is also provided.

### FIELD OF THE INVENTION

The present invention generally relates to the field of personal care formulations for the skin, and more particularly to a low viscosity oil-in-water emulsion which is stable despite having a high proportion of oil. The invented formulations are suitable for application to all skin, with several embodiments described herein being configured to be particularly beneficial for hard-to-reach areas like the scalp. A method of formulating said composition as a low viscosity stable emulsion is also provided.

### BACKGROUND OF THE INVENTION

Many types of products for moisturizing the skin have been commonly available for many years. As the science of hydrating skin has evolved, the concept of hydration using molecules that are naturally found in nourished skin, known broadly as Natural Moisturizing Factors (NMFs), has gained prominence. NMFs are generally known as a wide range of naturally occurring building blocks for the skin. NMFs include components as diverse as amino acids, ceramides, phospholipids, saccharides, glycerin, hyaluronic acid, sodium pyrrolidine carboxylic acid (sodium PCA), urea, triglycerides, and fatty acids.

A particularly neglected area of the skin is the scalp. While the skin on the scalp is similar to skin on other parts of the body, it has more lipid glands and sweat glands, and a lower barrier function, resulting in less ability to maintain and replenish moisture. Scalps are therefore prone to moisture imbalances, which may manifest in many cases as dandruff. However, even in the absence of dandruff, it is important to keep the scalp nourished and healthy. The scalp provides support for follicles and the health of the hair emerging therefrom.

Existing scalp care formulations are typically formulated to have a relatively high viscosity, in order to maintain sufficient contact of the active ingredients with the scalp, and to prevent the formulation from running or dripping after application. They also typically include active ingredients such as zinc pyrithione or selenium sulfide, which are effective to control dandruff. These are relatively harsh active ingredients that can be irritating to the scalps of some consumers. In the absence of dandruff, there is little benefit to using scalp care formulations having such strong actives.

It would be beneficial to provide a scalp care formulation which nourishes the sensitive skin of the scalp without use of harsh active ingredients, and which avoids problems with applied excess product dripping during use. As well, because scalp formulations are typically used sparingly by the consumer, with only a small volume of product needing to be applied for a single use, the product will need to withstand long periods of storage and repeated use after being opened. Stability is therefore important to commercial marketability. For a scalp care formulation, it will be beneficial to manufacture a product that does not separate into its constituent oil and water phases during multiple phases of shipping under potentially extreme and varied conditions of temperature, storage, retailing, and use.

### SUMMARY OF THE INVENTION

The skincare formulation of the invention is formulated to have a low viscosity, having a thickness similar to that of water. This feature facilitates fast absorption into the skin of areas like the scalp, which in turn obviates the need to have a thicker formulation.

It is further formulated as an oil-in-water emulsion having a high percentage of an emollient, which is surprisingly stable despite the presence of emollient at such a high level. A particularly preferred emollient is squalane. Squalane is a beneficial moisturizing hydrocarbon oil which is efficacious for addressing dry skin, particularly on the scalp. It has a dry feel and also absorbs quickly, preventing any problematic accumulation of residue on the scalp or hair.

A method of formulating the composition is also provided which utilizes specialized equipment to reduce particle sizes of the oil phase. Examples of such equipment which may be used are homogenizers, wet mills, or microfluidizers. Microfluidizers are particularly effective for this application. Microfluidizers may be used for high-pressure homogenizations, in order to make nano-emulsions by reducing the sizes of suspensions and emulsions. In this case, the microfluidizer may be used to process the invented formulation into a low viscosity, stable emulsion. Further, the use of a microfluidizer makes it possible to prepare the invented formulation as a stable oil-in-water emulsion, rather than a water-in-oil emulsion, despite the high levels of oil. The method overcomes significant technical challenges with working with high percentages of oil-based ingredients.

Accordingly, in one aspect of the invention, a personal care formulation consisting of an oil-in-water emulsion is provided comprising emollient at a high percentage of 15.00-20.00%, most preferably 15.00%.

In another aspect of the invention, a personal care formulation consisting of an oil-in-water emulsion is provided which includes natural moisturizing factors including hyaluronic acid, amino acids, and sodium PCA.

In another aspect of the invention, a formulation is provided comprising an oil-in-water emulsion having a viscosity close to that of water.

In a further aspect of the invention, a formulation is provided having excellent freeze-thaw stability, despite being an oil-in-water emulsion with a particularly high proportion of oil.

In a still further aspect of the invention, a method of formulating a personal care composition having a high concentration of emollient and which provides a thin emulsion having a viscosity similar to that of water is provided.

In a still further aspect of the invention, a use is provided of such a personal care composition for the treatment of the skin or other areas in need of moisture, in particular dry skin, hair, eyelashes, eyebrows, nails, or scalp.

In a still further aspect of the invention, a process for the nontherapeutic treatment of the skin or other areas in need of moisture, in particular dry skin, hair, eyelashes, eyebrows, nails, or scalp is provided, which comprises applying, to the substrate, the personal care composition of the invention.

Furthermore, as described herein, the emulsions obtained according to the invention remain stable and homogenous over extended times at various temperatures, and even under conditions of repeated freezing and thawing.

There are a number of advantages to the present invention. The present cosmetic composition provides the consumer with an easy-to-apply leave-on personal care formulation containing high levels of an emollient in addition to other NMFs, which all serve to nourish the skin. The formulation is made as an oil-in-water emulsion and has a low viscosity, despite the high levels of emollient. It also does not tend to have a heavy feel on the scalp or hair, or any substrate to which it has been applied. The lower viscosity means that the formulation can be applied and quickly absorbed by the scalp, with less likelihood of over-application of product and subsequent dripping or running. The formulation is also highly stable at a range of incubation times at various temperatures. Thus, the invention provides a beneficial balance between efficacy, ease of use, coverage, and stability.

These and other aspects of the invention will become apparent from the description that follows.

### DETAILED DESCRIPTION OF THE INVENTION

Provided herein is a novel skin-care formulation, which is beneficial for all skin but is particularly beneficial for the scalp, and which despite being formulated as an oil-in-water formulation with a high percentage of emollient, constitutes a low viscosity formulation. This results in a formula that is less likely to be applied in excess to areas such as the scalp, being quickly absorbed upon application. The formulation also shows strong features of stability under various extreme conditions, particularly for an oil-in-water emulsion having such a high level of oil.

The preferred embodiments of the formulation of the invention also comprise a number of NMFs, which are intended to mimic those components naturally found in healthy and nourished skin. The NMFs provided in the sample formulations are largely water-soluble components and are therefore distributed in the aqueous phase of the oil-in-water formulation.

A preferred emollient for use in the invention is squalane, which is the hydrogenated form of squalene which is found naturally in the skin. Squalane is advantageous for use in externally applied skin care formulations as the hydrogenated form is stable, whereas the non-hydrogenated squalene is prone to oxidation.

In a preferred embodiment of the formulation, squalane is provided at a relatively high percentage of up to 20.00% by weight, preferably up to 18.00% by weight, and most preferably up to 15.00% by weight. This is significant, as the formulation is prepared as a low viscosity, stable oil-in-water formulation. Typical oil-in-water formulations having a low viscosity will require a lower percentage of emollient. An emollient in the range of 5% and no more than 8% would be typical for such a thin emulsion that is proven stable. As described below, the 15% level of squalane in a remarkably stable formulation requires additional technically advanced processing steps to achieve. The inventors are not aware of any commercially offered oil-in-water thin emulsions which have achieved the level of stability described below for the invented formulation, while containing such a high percentage of oil.

A formulator seeking to include such a high level of oil would normally be required to prepare a water-in-oil emulsion, or to use other complex methods of emulsion. However, preparing a water-in-oil emulsion would not be a preferred option, especially for a formulation to be used on areas like the scalp. Water-in-oil emulsions have a heavier feel to the product. Common examples are night creams which tend to feel rich, heavy, and oily. For other skin care products and especially for a scalp product, it would be important to have a lighter feel and less tendency to accumulate on the surface of the scalp and hair, as is offered by oil-in-water emulsions.

Embodiments of the present invention are described as follows, which should not to be construed as limiting.

### Formulation Method

For formulating compositions of the present invention, most industrial homogenizing mixers (“kettles”) which are sized and configured to mix personal care formulations will be suitable. The kettle needs to be equipped with standard features such as interior side wall scrapers (also called sweepers) that are separately controllable from the homogenizing paddles.

A formulation of the invention can be prepared in three separate core phases (Phases A, B, and C), with optional additional ingredients for some of the phases, as described below. All steps described below may be performed in a room temperature environment. All amounts for each ingredient are provided as percentage of weight in the final formulation.

Phase A may be prepared by first adding water to a stainless steel kettle with built-in side sweepers and homogenizing paddles. An amount of water such that the final formulation has water in the range of 65-85% by weight is appropriate. As seen in a preferred sample formulation N1, the resulting weight percentage of water in the final formulation after combination of all of phases A, B, and C is 74.374884%. While the amounts provided in the manufacturing protocol provided below are described for sample formulation N1, the same procedures may be used to make any of the sample formulations outlined in this application.

In Phase A, if being used in the formulation, any number of water-soluble ingredients which contain NMFs may be added into the water. These ingredients will serve to nourish the scalp or skin when applied as part of the final formulation. In the example described below, four distinct water-soluble NMF-containing ingredients are used, and together add up to 6.53% of the final formulation by weight, however, given the relatively large volume of water, it has been noted that amounts of NMF-containing aqueous ingredients up to a total of 20.00% by weight could be used and would not present solubility difficulties. As well, more or fewer NMF-containing ingredients may be used.

If used, each aqueous NMF-containing ingredient is added one at a time, with mixing starting at low speed after addition of each in order to avoid foaming. Speed ranges of the homogenizer are generally set out below in respect of each stage of addition of further ingredients, but the skilled person will be able to use judgment to adjust speeds depending on the ingredient being added and the condition of the mixture as it is stirred. The skilled person will also be aware of what is considered a “high”, “medium”, or “low” speed in accordance with the model of homogenizer being used, and will be able to adapt the speed of homogenization accordingly.

To make the sample formulation as set out in Table 1 below, in Phase A, a modified hyaluronic acid-containing ingredient consisting of C12-13 alkyl glyceryl hydrolyzed hyaluronate, marketed by Kewpie Corporation under the name HYALO-REPAIR, is added to the water in the kettle, in an amount corresponding to 0.05% of the final formulation by weight. HYALO-REPAIR is promoted for cosmetic products as it facilitates moisture retention and barrier function recovery. It is mixed into the water starting at a low speed to avoid foaming, under homogenization and with the side sweepers activated, until the batch becomes colourless and clear.

Next, a further NMF-containing ingredient called PRODEW 500 is added. PRODEW 500 is marketed by Ajinomoto Corporation, and contains NMFs such as sodium PCA and a number of amino acids. It is promoted as being a beneficial hydrating ingredient for hair care and other cosmetic products. The PRODEW 500 may be added in an amount corresponding to 5.00% of the formulation by weight, and should be mixed in at a low speed to facilitate dispersal within the batch. The batch is mixed at low speed, with side sweepers activated, until the batch is colourless and clear.

A third NMF-containing ingredient may then be added, called HYGROPLEX V, a hair conditioning active ingredient marketed by CLR. An amount equivalent to 1.00% of the final formulation may be added. This ingredient contains hexylene glycol, sugars, and amino acids. It is also added at a low speed, under homogenization and with the side sweepers activated. Mixing occurs until the batch is colourless and clear.

A final NMF-containing ingredient, PHYSIOGENYL PF, marketed by Solabia, may then be added at an initial low speed, with the homogenizer speed eventually being increased, and side sweeper activated while avoiding the generation of foam. This ingredient contains several salts of PCA, and is promoted as facilitating effective hydration of the skin. This may be added in an amount corresponding to 0.48% of the final formulation by weight. Mixing should occur until the batch is colourless and clear.

After incorporation of the final water-soluble NMF-containing ingredient, phase A should be continued to be mixed at a higher speed, with a moderate speed for the side sweepers, with periodic samples of the mixture being taken from the bottom of the kettle (also known as valve purges or bucket flips) as needed to ensure sufficient homogenization. The mixing should continue until phase A presents as uniform, clear and colourless. The viscosity of the fully mixed phase A remains low, being similar to that of water.

Phase B is then mixed in a separate kettle. Phase B is a mixture made of hydrophobic or oil phase-compatible ingredients such as the emollient, preservative, and emulsifier. In the below sample formulation, the emollient used is squalane in an amount corresponding to 15.00% by weight of the final formulation (NEOSSANCE squalane may be used). However, amounts of emollient up to 20.00% can also be used although there will be some minor amount of emollient that may not be fully emulsified as will be explained further below. While squalane is used in the preferred embodiment, many other emollients appropriate for personal care formulations could be substituted in whole or in part for the squalane, such as caprylic/capric triglyceride or coco caprylate.

At least one surfactant is also necessary to make an emulsion. A broad range of surfactants (also often termed emulsifiers) are available in the field. In a preferred embodiment, the inventors utilized a combination of two surfactants. First, isoceteth-20 (BRIJ IC20-70) marketed by Croda may be also added to Phase B in an amount corresponding to 1.00% of the final formulation by weight. A second surfactant may also be used in the preferred formulation in Table 1, FLUIDIFEEL EASY (lauryl glucoside, myristyl glucoside, and polyglyceryl-6-laurate). This ingredient is marketed by Seppic. It may be incorporated into Phase B in an amount corresponding to 2.00% of the final formulation by weight.

Many surfactants may be used in substitution for BRIJ IC20-70 and FLUIDIFEEL EASY. It is preferred to use surfactants that are liquid at room temperature so that an additional step of melting or solubilizing the surfactant into the formulation is not necessary. However, at a low temperature, even a normally liquid surfactant such as FLUIDIFEEL EASY may crystallize. If this happens, it may be heated and mixed until homogenous.

An optional ingredient used in the preferred formulation is a preservative. In the above example, an amount of ISCAGUARD CPP (chlorphenesin and phenoxyethanol) marketed by ISCA is then added to Phase B in an amount corresponding to 1.00% of the final formulation. An amount in the range of 0.90-1.1% would also be effective. Many other preservatives for personal care formulations are also suitable for use in the invention in substitution for ISCAGUARD CPP.

To prepare Phase B, in the case of the above specific ingredients, it is preferable to make a premixture of BRIJ IC20-70, FLUIDIFEEL EASY, and ISCAGUARD CPP before adding the final ingredient, NEOSSANCE SQUALANE. It should be mixed gently until uniform, using only the side sweepers. Phase B when thoroughly mixed presents as a translucent to transparent colourless liquid with a viscosity similar to that of water.

After Phase A and Phase B are both homogenous, the next step is to add Phase B slowly and steadily to Phase A, while continually mixing at high speed using both the homogenizer and the side sweepers. Milling at a moderate frequency is then performed. Mixing and milling for approximately 5-10 minutes under these conditions is suitable. The resulting batch is emulsified, having an appearance which is translucent to opaque white, with a viscosity similar to that of water.

Phase C is optional. When used, it may consist of a single ingredient used to adjust pH. A suitable ingredient is PURAC ULTRAPURE 90 marketed by Corbian, which consists of lactic acid and water. In the sample formulation, Phase C is included an amount corresponding to 0.075116% by weight of PURAC ULTRAPURE 90 being mixed into the combined Phase A and B, and mixed at a high speed for the homogenizer and moderate speed for the side sweepers, along with mill recirculation if deemed necessary. Amounts of PURAC ULTRAPURE 90 in the range of 0.067-0.083 would also work well in the invented formulation. As well, a number of other pH adjusting acids would work well in the formulation.

The product now appears as a translucent to opaque thin white emulsion. If Phase C is included, the product should have a pH in a desirable range of 4.00-5.00 which is suitable for skin care products.

A further extended mixing is then conducted in order to ensure that the batch containing Phases A, B, and C (if used) is uniformly mixed. Using high speeds for the homogenizer and moderate speed for the side sweepers, the product is stirred for approximately 30 minutes.

At this point the product should be further processed in order to reduce the size of the particles of the emulsion. There are several means for conducting such processing. For instance, one may use standard mills appropriate for cosmetic formulations. There are many sources for such mills, for example the colloid mill product line available from Probst & Class. There are also suitable sonicators available from Sonic Corporation.

The batch may also be processed using a microfluidizer, which is a high pressure homogenizer used to perform micro-mixing of emulsions and dispersions. While continuing to be gently mixed, the batch is pushed through the microfluidizer at high pressure at 10,000-30,000 psi, through thin pipe channels to minimize the particle size of the formulation. The most preferred pressure is at 30,000 psi if this setting is available on the equipment being used, but the range of 10,000-25,000 psi still produces satisfactory product. A microfluidizer used by the inventors with satisfactory results was the MICROFLUIDICS M110P V3 Microfluidizer Processor, manufactured by Microfluidics Corp. of Westwood, MA. Other models of microfluidizer from MICROFLUIDICS and other sources may also be used to achieve the same result. The microfluidizer should be set with as short a recirculation loop as is available, and a high rate of flow to avoid opportunities for separation of the product.

If using a microfluidizer, after the first run through, the batch may then be processed a second time and third time through the microfluidizer. The formulation is then complete. It is not essential to process the batch three times through the microfluidizer, as the first run will be generally sufficient to result in a stable formulation. However, to completely ensure thorough processing of the formulation, two or more rounds of microfluidization are recommended.

The sample formulation which the above method was used to prepare is shown in Table 1.

TABLE 1
Formulation N1

| Ingredient Trade | | |
|---|---|---|
| Name | Ingredients | % (w/w) |
| D.I. WATER | Water | 74.394884 |
| HYALO-REPAIR | C12-13 Alkyl Glyceryl Hydrolyzed Hyaluronate | 0.05% |
| PRODEW 500 | Sodium PCA (and) Sodium Lactate (and) Arginine (and) | 5.00% |
| | Aspartic Acid (and) PCA (and) Glycine (and) Alanine (and) | |
| | Serine (and) Valine (and) Proline (and) Threonine (and) | |
| | Isoleucine (and) Histidine (and) Phenylalanine | |
| HYGROPLEX V | Hexylene Glycol (and) Water (and) Fructose (and) Glucose | 1.00% |
| | (and) Sucrose (and) Urea (and) Dextrin (and) Alanine (and) | |
| | Glutamic Acid (and) Aspartic Acid (and) Hexyl Nicotinate | |
| PHYSIOGENYL PF | Water (and) Sodium PCA (and) Magnesium PCA (and) Zinc | 0.48% |
| | PCA (and) Phenoxyethanol (and) Manganese PCA (and) | |
| | Potassium Sorbate | |
| NEOSSANCE | Squalane | 15.00% |
| SQUALANE | | |
| BRIJ IC20-70 | Isoceteth-20 | 1.00% |
| FLUIDIFEEL | Lauryl Glucoside & Myristyl Glucoside & Polyglyceryl-6 | 2.00% |
| EASY | Laurate | |
| ISCAGUARD CPP | Chlorphenesin (and) Phenoxyethanol | 1.00% |
| PURAC | Lactic Acid, Aqua | 0.075116% |
| ULTRAPURE 90 | | |

Following microfluidization, the resulting formulation is a translucent to opaque emulsion, with a pH in the range of 4.0-5.0, and with a viscosity close to that of water. Despite the high amount of emollient included, the emulsion made in accordance with the described example is thin, having an average viscosity of 5.26 centipoise (cP). For context, the viscosity of water is 1 cP, the viscosity of a typical face cream is 255,000 cP, and of a hand cream is 465,000 cP. The viscosity of the formulation is therefore low and close to that of water. Viscosity measurements were taken in standard conditions at room temperature (25° C.) using a BROOKFIELD DVEELVTJ0 digital viscometer, with its spindle and speed set at LVT 61, 100 RPM. Ten measurements of N1 samples taken in the above conditions are set out in Table 1 below.

TABLE 2

| | Viscosity |
|---|---|
| Sample | (centiPoise) |
| N1-1 | 4.92 cP |
| N1-2 | 4.86 cP |
| N1-3 | 4.92 cP |
| N1-4 | 4.98 cP |
| N1-5 | 5.16 cP |
| N1-6 | 5.40 cP |
| N1-7 | 5.58 cP |
| N1-8 | 5.40 cP |
| N1-9 | 6.06 cP |
| N1-10 | 5.28 cP |

Further measurements of formulations and batches of the invention have shown consistent viscosities in the above conditions of 4.0 to 8.0 cP, most frequently in the range of 4.5 to 6.0 cP.

In terms of density, the specific gravity of the resulting formulation is in the range of 0.95-1.05. Following two runs through the microfluidizer, the particle size in the emulsion has a DV50 (median for the volume distribution) range of equal to or greater than 100-300 nm. In other words, DV50 is not less than 100 nm.

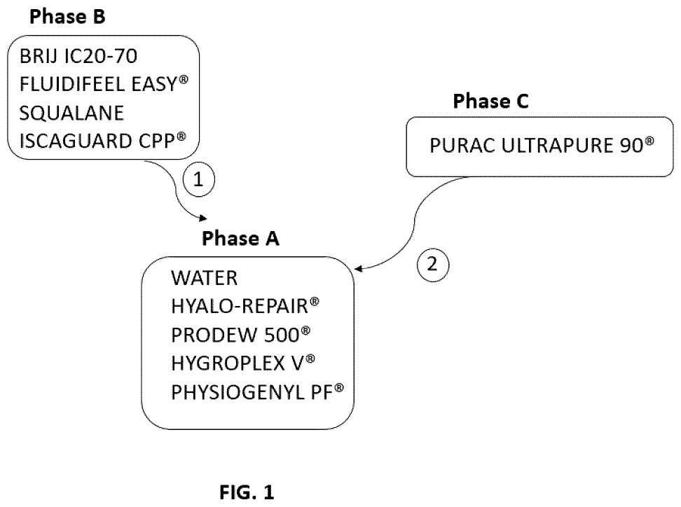

An overview of the core process of combining phases A, B, and C in the case of the formulation of Table 1 is also shown in FIG. 1.

It should be noted that depending on the amount of emollient and surfactant used, some of the resulting final formulations may show signs of a small portion of the oil phase not being completely emulsified. While the preferred formulation of Table 1 and others as further detailed below in Table 5 (Category A) are completely emulsified, some formulations having higher amounts of emollient were observed to have some oil phase droplets visible at the top of the emulsion, observed immediately after formulation (see Categories B and C). Oil phase droplets appearing on top could be distinguished from situations in which the oil phase separated from the aqueous phase, which occurs after the time of formulation, and can be observed as an oil layer which begins forming at the bottom of the emulsion and grows as time passes.

Even in those formulations with oil droplets on top, the emulsions still remained stable for varying periods of time as described in the stability test results below.

A small amount of oil droplets appearing on the formulation is not problematic for commercial marketability, as users are accustomed to shaking cosmetic products prior to use to ensure even distribution of components in an aliquot dispensed for a single use. However, if a complete and entirely homogenous emulsion is desired, amounts of emollient and surfactant should be chosen within the Category A ranges as detailed below.

The formulations of the invention may further include additional ingredients, subject to solubility in one of Phases A or B, as may be ascertained by the skilled person. Such additional ingredients may include cosmetic ingredients that enhance or supplement a skin care formulation. The term “cosmetic ingredient” is used herein to refer to a product for application to the body (e.g. skin, hair) to improve or enhance the skin. Thus, additional cosmetic ingredients may be included such as conditioners, humectants, silicones, and sunscreens. Other examples of cosmetic ingredients include, but are not limited to, peptides; other amino acids; carbohydrates (e.g. ascorbyl glucoside, glucosyl hesperidin, and saccharide isomerate); proteins (e.g. collagen, elastin, and hydrolyzed lupine protein); lipids (e.g. ceramides, glycosphingolipids, and phospholipids); vitamins (e.g. niacinamide, biotin, ascorbic acid, retinol, retinoic acid, and panthenol); benzyl alcohol; sterols (e.g. phytosterols, punica granatum sterols, and 7-dehydrocholesterol); glycols; glycolic acid; iso cyclomone; allantoin; PEGs; flavanoids (e.g. apigenin, heseperidin, and genistein); esters (e.g. methylsilanol mannuronate, butyl avocadoate, and jojoba esters); botanical products (e.g. Tasmannia lanceolata fruit/leaf extract, Curculigo orchioides root extract, and rose flora); keratolytic agents such as salicylic acid; betaines; glutamates; xylitols; sorbitol; caffeine; menthol; menthyl lactate; zinc oxide; zinc carbonate; erythritol; glycerin; lactates; and biotechnological products (e.g. Pseudoalteromonas exopolysaccharides, Alteromonas ferment filtrate, sodium hyaluronate).

The formulation of the invention may also contain vitamins and derivatives thereof such as panthenol, ascorbic acid, niacinamide, tocopherol; anti-inflammatory ingredients such as allantoin, phytantriol, sphingosine and bisabolol; other moisturizing ingredients such as trimethylglycine and polyglutamic acid; additional antioxidants such as flavonoids, xanthones, isoflavones, alpha-lipoic acid; and other anti-aging actives such as beta-glucans, alpha hydroxy acids, and beta hydroxy acids.

The formulation may also include fragrance such as essential oils, plant extracts, or synthetic fragrance. Examples include vanilla, cucumber, aloe vera, almond, mango, coconut, melon, linalool, citronellol, cinnamol, limonene, geraniol, eugenol, lavender oil, rose flower extract, bergamot oil, ylang-ylang oil, lemon, lime, orange, tangerine, peppermint, spearmint, and eucalyptus.

As used herein, the term “about” refers to an amount within 10% or less of the indicated amount, and preferably an amount within 5% or less, wherein the amount is either greater or less than the indicated amount.

The present invention corresponds to a skin care formulation comprising a low viscosity oil-in-water emulsion made with a method which includes additional processing to reduce particle size, and containing, as percentages by weight of the final formulation:
(a) an emollient in the amount of 15.00-20.00%, more preferably about 15.00%;
(b) one or more surfactants in the amount of 2.00-4.50%, more preferably 2.50-3.50%, and still more preferably about 3.00%;
(c) water;
the proviso being that (a) to (c) must add up to 100%.

The present invention also corresponds to a skin care formulation comprising an oil-in-water emulsion containing, as percentages by weight of the final formulation:
(a) an emollient in the amount of 15.00-20.00%, more preferably about 15.00%;
(b) one or more surfactants in the amount of 2.00-4.50%, more preferably 2.50-3.50%, and still more preferably about 3.00%;
(c) water-soluble NMF-containing ingredients in the amount of 0.05-20.00%, more preferably 0.05-10.00%; and
(d) water;
the proviso being that (a) to (d) must add up to 100%.

The present invention also corresponds to a skin care formulation comprising an oil-in-water emulsion containing, as percentages by weight of the final formulation:
(a) an emollient in the amount of 15.00-20.00%, more preferably about 15.00%;
(b) one or more surfactants in the amount of 2.00-4.50%, more preferably 2.50-3.50%, and still more preferably about 3.00%;
(c) water-soluble NMF-containing ingredients in the amount of 0.05-20.00%, more preferably 0.05-10.00%;
(d) a preservative in the amount of 0.90-1.1%, preferably about 1.0%; and
(e) water;
the proviso being that (a) to (e) must add up to 100%.

The present invention also corresponds to a skin care formulation comprising an oil-in-water emulsion containing, as percentages by weight of the final formulation:
(a) an emollient in the amount of 15.00-20.00%, more preferably about 15.00%;
(b) one or more surfactants in the amount of 2.00-4.50%, more preferably 2.50-3.50%, and still more preferably about 3.00%;
(c) water-soluble NMF-containing ingredients in the amount of 0.05-20.00%, more preferably 0.05-10.00%;
(d) a preservative in the amount of 0.90-1.1%, preferably about 1.0%; and
(e) a pH adjustor in the amount of 0.067-0.083%, preferably about 0.075%; and
(f) water;
the proviso being that (a) to (f) must add up to 100%.

The present invention also corresponds to a skin care formulation comprising an oil-in-water emulsion containing, as percentages by weight of the final formulation:
(a) an emollient in the amount of about 15.00%;
(b) one or more surfactants in the amount of 2.00-4.50%, more preferably 2.50-3.50%, and still more preferably about 3.00%;
(c) water-soluble NMF-containing ingredients in the amount of about 6.53%;
(d) a preservative in the amount of about 1.0%; and
(e) a pH adjustor in the amount of about 0.075%; and
(f) water q.s.

The present invention also corresponds to a skin care formulation comprising an oil-in-water emulsion containing, as percentages by weight of the final formulation:
(a) squalane in the amount of about 15.00%;
(b) one or more surfactants in the amount of 2.00-4.50%, more preferably 2.50-3.50%, and still more preferably about 3.00%;
(c) water-soluble NMF-containing ingredients chosen as some or all of the following: HYALO-REPAIR; PRODEW 500, HYDROPLEX V, and PHYSIOGENYL PF, in the total amount of about 6.53%;
(d) a preservative in the amount of about 1.0%; and
(e) a pH adjustor in the amount of about 0.075%; and
(f) water q.s.

The present invention also corresponds to the above formulations wherein said formulations have high stability in a variety of conditions of time and temperature.

The present invention also corresponds to the above formulations wherein the pH is in the range of 4.0-5.0.

The present invention also corresponds to the above formulations wherein said formulations have low viscosity, having viscosities close to that of water.

The present inventions also corresponds to the above formulations having viscosities in the range of 4.5-8 centiPoise, more preferably 5-7 centiPoise.

The present invention also corresponds to a method of manufacturing the above formulations using a process including means of reducing the particle size of the oil phase of the emulsion.

The present invention also corresponds to the above formulations when made in accordance with the methods described herein.

The present invention also corresponds to use of the above formulations to moisturize the skin, hair, eyelashes, eyebrows, nails, and/or scalp.

The present invention also corresponds to use of the above formulations to moisturize the scalp.

The formulations of the invention are for use on the skin after cleansing by rubbing a small amount directly onto the skin. It is particularly suited for use on the scalp, but may be used on any external surface of the skin or hair.

### Viscosity

As described above, the invented formulations have a high percentage of emollient and are oil-in-water emulsions. With such a high percentage of emollient, in order to make a stable emulsion, it is typically required to make a water-in-oil emulsion instead. As mentioned previously, this would be undesirable due to the heavier and more oily feel of water-in-oil emulsions. The inventors were able to achieve a highly stable emulsion through careful control of the ratios of surfactant to emollient, and through use of further processing steps in the manufacturing process to reduce particle sizes of the oil phase.

It is also a significant technical advance that the formulation is a thin emulsion, having a viscosity close to that of water. When emollients are included at such a high percentage, the resulting emulsion is generally much thicker and denser, and usually required to take the form of creams or gels.

As mentioned above, the low viscosity of the emulsion is advantageous for ease of application of the formulation to hard-to-reach areas like the scalp. The low viscosity means that the product can be appropriately applied in a small volume, absorbs quickly into the skin or scalp, and excess product does not accumulate on the surface of the skin or scalp.

### Efficacy

The formulation was confirmed to be cosmetically acceptable, in that the combination of components are suitable for use in contact with keratinous tissue without toxicity or incompatibility. The formulation also underwent clinical testing to determine its effectiveness in skin hydration. The formulation N1 as set out in Table 1 above was tested for its ability to reduce water loss from the skin surface, using the internationally accepted standard Transepidermal Water Loss (TEWL) testing protocol. TEWL is a very sensitive indicator of skin barrier integrity, which is measured instrumentally by evaporometry. TEWL refers to the total share of water which is lost from the dermal and epidermal tissues to the outside through the stratum corneum. Decreases in the integrity of skin barrier function are indicated by elevated levels of TEWL, while improvements to the skin barrier are indicated by decreases in TEWL. To measure using the TEWL protocol, the water evaporating from the skin may be measured using an evaporometer having a probe that is placed in contact with the skin surface, containing sensors that detect changes in water vapor release.

The TEWL testing was performed on the scalps of 53 healthy volunteers who were between 18 to 65 years old, including a sampling of different ethnicities and hair types. TEWL was measured using an evaporometer, which measures water vapor released from a surface in g/h/m². The evaporometer used was the Tewameter™ Nano, manufactured by Courage-Khazaka GmbH of Germany.

Before the beginning of the testing, subjects were instructed to wash their hair over a one week period with a standard shampoo provided by the clinic, and to use no other hair products. The Time 0 (To) TEWL measurement was then taken under standardized conditions in an air-conditioned room, after a fifteen-minute relaxation period for the subject so as to avoid anomalies in sampling due to sweating or stress. The subject was then given standard shampoo and conditioner to take home and was instructed to wash and condition their hair on their usual schedule, and to apply a few drops of the invented formulation on a daily basis to a clean dry, scalp, massaging the product into the scalp for 30 seconds, and then leaving the product to absorb, without rinsing.

TEWL values for each of the 53 subjects were also taken after daily use of the product for two weeks with an allowance of +/−two days (T_{2weeks}), and after four weeks of product use with an allowance of +/−two days (T_{4weeks}). At the end of the study, the amount of product used by each subject was recorded by weighing the sample of the test products to record overall usage by comparing it to the weight of the full amount of product provided at the beginning of the study. All participants were found to have used similar amounts.

The measurements for all 53 subjects were as shown in Table 3 below.

TABLE 3

| # | T0 | T2 wks | T4 Wks | T2 − T0 | T4 − T2 | T4 − T0 |
|---|---|---|---|---|---|---|
| 1 | 17.6 | 16.9 | 10.4 | −0.7 | −6.5 | −7.2 |
| 2 | 32.5 | 22.6 | 22.3 | −9.9 | −0.3 | −10.2 |
| 3 | 38.3 | 20.1 | 27 | −18.2 | 6.9 | −11.3 |
| 4 | 21.7 | 24 | 25.2 | 2.3 | 1.2 | 3.5 |
| 5 | 38.1 | 32.1 | 31.4 | −6 | −0.7 | −6.7 |
| 6 | 30.8 | 33.3 | 32.4 | 2.5 | −0.9 | 1.6 |
| 7 | 17.7 | 16.1 | 18.9 | −1.6 | 2.8 | 1.2 |
| 8 | 39.3 | 26.8 | 24.3 | −12.5 | −2.5 | −15 |
| 9 | 10.6 | 9.7 | 11.0 | −0.9 | 1.3 | 0.4 |
| 10 | 19.7 | 11.9 | 15.1 | −7.8 | 3.2 | −4.6 |
| 11 | 18.6 | 19.0 | 11.7 | 0.4 | −7.3 | −6.9 |
| 12 | 18.4 | 21.2 | 18.5 | 2.8 | −2.7 | 0.1 |
| 13 | 25.8 | 19.5 | 22.8 | −6.3 | 3.3 | −3 |
| 14 | 23.5 | 17.6 | 16.4 | −5.9 | 1.2 | −7.1 |
| 15 | 17.4 | 15.6 | 19.2 | −1.8 | 3.6 | 1.8 |
| 16 | 52.8 | 50.2 | 49.2 | −2.6 | −1 | −3.6 |
| 17 | 35.5 | 20.7 | 14.0 | −14.8 | −6.7 | −21.5 |
| 18 | 30.0 | 22.2 | 21.9 | −7.8 | −0.3 | −8.1 |
| 19 | 28.7 | 27.8 | 27.4 | −0.9 | −0.4 | −1.3 |
| 20 | 25.5 | 22.6 | 16.9 | −2.9 | −5.7 | −8.6 |
| 21 | 22.3 | 19.1 | 12.7 | −3.2 | −6.4 | −9.6 |
| 22 | 21.6 | 18.9 | 25.1 | −2.7 | 6.2 | 3.5 |
| 23 | 40.5 | 23.8 | 20.4 | −16.7 | −3.4 | −20.1 |
| 24 | 25.7 | 22.0 | 17.3 | −3.7 | −4.7 | −8.4 |
| 25 | 21.8 | 15.3 | 23.4 | −6.5 | 8.1 | 1.6 |
| 26 | 24.7 | 23.1 | 22.8 | −1.6 | −0.3 | −1.9 |
| 27 | 35.7 | 15.1 | 22.4 | −20.6 | 7.3 | −13.3 |
| 28 | 20.5 | 22.8 | 17.1 | 2.3 | 5.7 | −3.4 |
| 29 | 16.1 | 11.8 | 17.4 | −4.3 | 5.6 | 1.3 |
| 30 | 46.9 | 42.3 | 41.5 | −4.6 | −0.8 | −5.4 |
| 31 | 20.0 | 12.7 | 13.4 | −7.3 | 0.7 | −6.6 |
| 32 | 14.2 | 11.3 | 8.9 | −2.9 | −2.4 | −5.3 |
| 33 | 25.8 | 23.1 | 27.3 | −2.7 | 4.2 | 1.5 |
| 34 | 16.8 | 18.0 | 19.3 | 1.2 | 1.3 | 2.5 |
| 35 | 23.2 | 20.5 | 25.6 | −2.7 | 5.1 | 2.4 |
| 36 | 17.0 | 17.6 | 11.5 | 0.6 | −6.1 | −5.5 |
| 37 | 21.2 | 20.4 | 17.8 | −0.8 | −2.6 | −3.4 |
| 38 | 19.7 | 12.2 | 20.6 | −7.5 | 8.4 | 0.9 |
| 39 | 29.7 | 24.7 | 25.4 | −5 | 0.7 | −4.3 |
| 40 | 21.3 | 19.2 | 16.4 | −2.1 | −2.8 | −4.9 |
| 41 | 21.8 | 23.3 | 17.4 | 1.5 | −5.9 | −4.4 |
| 42 | 31.3 | 20.8 | 9.4 | −10.5 | −11.4 | −21.9 |
| 43 | 16.8 | 18.1 | 12.8 | 1.3 | −5.3 | −4 |
| 44 | 28.6 | 17.1 | 17.2 | −11.5 | 0.1 | −11.4 |
| 45 | 71.3 | 32.5 | 21.7 | −38.8 | −10.8 | −49.6 |
| 46 | 24.0 | 18.0 | 22.9 | −6 | 4.9 | −1.1 |
| 47 | 18.1 | 16.2 | 12.0 | −1.9 | −4.2 | −6.1 |
| 48 | 18.4 | 11.9 | 16.3 | −6.5 | 4.4 | −2.1 |
| 49 | 25.1 | 26.7 | 18.3 | 1.6 | −8.4 | −6.8 |
| 50 | 16.6 | 18.7 | 10.7 | 2.1 | −8 | −5.9 |
| 51 | 15.3 | 14.3 | 16.4 | −1 | 2.1 | 1.1 |
| 52 | 17.1 | 13.7 | 13.2 | −3.4 | −0.5 | −3.9 |
| 53 | 19.8 | 16.1 | 9.0 | −3.7 | −7.1 | −10.8 |
| Averages | 25.50 | 20.59 | 19.62 | −4.91 | −0.97 | −5.88 |

The averaged results across all subjects were as shown in Table 4 below.

TABLE 4
Mean TEWL Values

| Time | TEWL (g/h/m²) | % Change in TEWL |
|---|---|---|
| T₀ | 25.5 | n/a |
| T2 weeks +/− 2 days | 20.6 | −19.2% |
| T4 weeks +/− 2 days | 19.6 | −23.1% |

As shown by these results, there was a 15-20% (average 19.6%) decrease in water loss from the scalp surface after two weeks of product use, and a 20-25% (average 23.1%) decrease in water loss from the scalp surface after four weeks of product use. These results were confirmed to be statistically significant by application of the Student t test at 95% confidence. It was therefore established that the invented formulation is efficacious in terms of hydration of the scalp and reducing evaporation of moisture therefrom.

Subjective comments from the participants on the invented formulation were also collected. 77.4% of participants felt satisfied or very satisfied with the product, and 75.5% of participants stated that their scalps felt hydrated after use of the product. The majority of these participants stated that their scalps felt noticeably hydrated by two weeks of use, with the remainder claiming that they felt hydration immediately, or after four weeks of use. Further, 86.8% assessed the product as easy to apply, and 79.2% stated that the product absorbed completely within 30 seconds after application. Overall, the comments on the usability and subjective effectiveness of the product were positive. The objective measurements of product efficacy as provided by the above TEWL results further confirm effectiveness of the product as a hydrator of the skin of the scalp.

### Additional Formulations

In addition to the formulation of Table 1 (N1), nine additional formulations with variations in the amounts of emollient and surfactants were prepared in accordance with the manufacturing procedure described above, which included two rounds of microemulsion. The additional formulations were then assessed for physical characteristics immediately afterwards. As shown below in Table 5, some formulations had complete incorporation of the oil phase into the emulsion (Category A), whereas some had varying amounts of oil droplets visible on top of the remaining ingredients which were otherwise fully emulsified (Categories B and C). As mentioned previously, the existence of some oil droplets would not preclude the formulation being used commercially. The pH of all formulations were in the acceptable range of 4.0-5.0.

The formulations can be placed into three categories: (A) fully emulsified; (B) small amount of oil phase not incorporated (small, under 1 mm in diameter, droplets of oil on the surface of the remaining ingredients); and (C) moderate amount of oil phase not incorporated (medium, over 1 mm in diameter, droplets of oil on the surface of the remaining ingredients). The formulations are shown in Table 5 below.

TABLE 5a
Formulations N1-N10

| | | | | | | | | Category B: | | Category C: | |
|---|---|---|---|---|---|---|---|---|---|---|---|
| | | | | | | | | White opaque, but | | White opaque, but | |
| | Physical | | | | | | | small (<1 mm | | medium (>1 mm | |
| | Characteristics | Category A: | | | | | | diameter) oil droplets | | diameter) oil droplets | |
| | Following | White opaque, uniform consistency | | | | | | visible on surface | | visible on surface | |
| | Formulation | Odourless | | | | | | Odourless | | Odourless | |
| | Sample | Water-like | | | | | | Water-like | | Water-like | |
| | Sources/ | pH 4.50-4.60 | | | | | | pH 4.50-4.60 | | pH 4.50-4.60 | |
| Ingredients | Trade Names | N1 | N4 | N5 | N3 | N8 | N7 | N2 | N6 | N9 | N10 |
| water | D.I. WATER | q.s. | q.s. | q.s. | q.s. | q.s. | q.s. | q.s. | q.s. | q.s. | q.s. |
| C12-13 Alkyl | HYALO- | 0.05 | 0.05 | 0.05 | 0.05 | 0.05 | 0.05 | 0.05 | 0.05 | 0.05 | 0.05 |
| Glyceryl | REPAIR | | | | | | | | | | |
| Hydrolyzed | | | | | | | | | | | |
| Hyaluronate | | | | | | | | | | | |
| Sodium PCA | PRODEW 500 | 5.00 | 5.00 | 5.00 | 5.00 | 5.00 | 5.00 | 5.00 | 5.00 | 5.00 | 5.00 |
| (and) Sodium | | | | | | | | | | | |
| Lactate (and) | | | | | | | | | | | |
| Arginine (and) | | | | | | | | | | | |
| Aspartic Acid | | | | | | | | | | | |
| (and) PCA | | | | | | | | | | | |
| (and) Glycine | | | | | | | | | | | |
| (and) Alanine | | | | | | | | | | | |
| (and) Serine | | | | | | | | | | | |
| (and) Valine | | | | | | | | | | | |
| (and) Proline | | | | | | | | | | | |
| (and) | | | | | | | | | | | |
| Threonine | | | | | | | | | | | |
| (and) Isoleucine | | | | | | | | | | | |
| (and) Histidine | | | | | | | | | | | |
| (and) | | | | | | | | | | | |
| Phenylalanine | | | | | | | | | | | |
| Hexylene | | HYGROPLEX | 1.00 | 1.00 | 1.00 | 1.00 | 1.00 | 1.00 | 1.00 | 1.00 | 1.00 |
| Glycol (and) | | V | | | | | | | | | |
| Water (and) | | | | | | | | | | | |
| Fructose (and) | | | | | | | | | | | |
| Glucose (and) | | | | | | | | | | | |
| Sucrose (and) | | | | | | | | | | | |
| Urea (and) | | | | | | | | | | | |
| Dextrin (and) | | | | | | | | | | | |
| Alanine (and) | | | | | | | | | | | |
| Glutamic (and) | | | | | | | | | | | |
| Acid (and) | | | | | | | | | | | |
| Hexyl | | | | | | | | | | | |
| Nicotinate | | | | | | | | | | | |
| Water (and) | PHYSIOGENYL | 0.48 | 0.48 | 0.48 | 0.48 | 0.48 | 0.48 | 0.48 | 0.48 | 0.48 | 0.48 |
| Sodium PCA | PF | | | | | | | | | | |
| (and) | | | | | | | | | | | |
| Magnesium | | | | | | | | | | | |
| PCA (and) Zinc | | | | | | | | | | | |
| PCA (and) | | | | | | | | | | | |
| Phenoxyethanol | | | | | | | | | | | |
| (and) | | | | | | | | | | | |
| Manganese | | | | | | | | | | | |
| PCA (and) | | | | | | | | | | | |
| Potassium | | | | | | | | | | | |
| Sorbate | | | | | | | | | | | |
| Squalane | NEOSSANCE, | 15.00 | 15.00 | 15.00 | 15.00 | 10.00 | 20.00 | 15.00 | 20.00 | 30.00 | 5.00 |
| | available from | | | | | | | | | | |
| | other sources | | | | | | | | | | |
| Isoceteth-20 | Surfactant such | 1.00 | 1.50 | 1.00 | 1.00 | 1.00 | 1.50 | 0.50 | 1.00 | 1.00 | 2.00 |
| | as BRIJ IC20-70, | | | | | | | | | | |
| | or other | | | | | | | | | | |
| | surfactants may | | | | | | | | | | |
| | be used | | | | | | | | | | |
| Lauryl | Surfactant such | 2.00 | 2.00 | 3.00 | 1.00 | 2.00 | 3.00 | 2.00 | 2.00 | 2.00 | 1.00 |
| Glucoside & | as FLUIDIFEEL | | | | | | | | | | |
| Myristyl | EASY, or other | | | | | | | | | | |
| Glucoside & | surfactants may | | | | | | | | | | |
| Polyglyceryl-6 | be used | | | | | | | | | | |
| Laurate | | | | | | | | | | | |
| Chlorphenesin | Preservative such | 1.00 | 1.00 | 1.00 | 1.00 | 1.00 | 1.00 | 1.00 | 1.00 | 1.00 | 1.00 |
| (and) | as ISCAGUARD | | | | | | | | | | |
| Phenoxyethanol | CPP, or other | | | | | | | | | | |
| | preservatives | | | | | | | | | | |
| | may be used | | | | | | | | | | |
| Lactic Acid | pH adjustor such | 0.075 | 0.075 | 0.075 | 0.075 | 0.075 | 0.075 | 0.075 | 0.075 | 0.075 | 0.075 |
| | as PURAC | | | | | | | | | | |
| | ULTRAPURE | | | | | | | | | | |
| | 90, or other acids | | | | | | | | | | |
| | may be used | | | | | | | | | | |

TABLE 5b
Formulations N1-N10 - Ratios of Emollient: Total Surfactants

| Ingredients | N1 | N4 | N5 | N3 | N8 | N7 | N2 | N6 | N9 | N10 |
|---|---|---|---|---|---|---|---|---|---|---|
| Squalane | 15.00 | 15.00 | 15.00 | 15.00 | 10.00 | 20.00 | 15.00 | 20.00 | 30.00 | 5.00 |
| Isoceteth-20 | 1.00 | 1.50 | 1.00 | 1.00 | 1.00 | 1.50 | 0.50 | 1.00 | 1.00 | 2.00 |
| Lauryl | 2.00 | 2.00 | 3.00 | 1.00 | 2.00 | 3.00 | 2.00 | 2.00 | 2.00 | 1.00 |
| Glucoside & | | | | | | | | | | |
| Myristyl | | | | | | | | | | |
| Glucoside & | | | | | | | | | | |
| Polyglyceryl-6 | | | | | | | | | | |
| Laurate | | | | | | | | | | |
| Ratio of | 15:3 | 15:3.5 | 15:4 | 15:2 | 10:3 | 20:4.5 | 15:2.5 | 20:3 | 30:3 | 5:3 |
| Emollient:Total | (5:1) | (4.3:1) | (3.8:1) | (7.5:1) | (3.3:1) | (4.4:1) | (6:1) | (6.7:1) | (10:1) | (1.7:1) |
| Surfactant | | | | | | | | | | |

In the above examples, all concentrations are listed as percentage weight of the final formulation, and may exclude subsidiary materials like diluents or fillers. The listed formulations therefore comprise the listed ingredients and such subsidiary materials as may be selected by a skilled person, depending on the physical and chemical characteristics of the remaining ingredients chosen for the composition.

It can be seen that in terms of physical characteristics, the inventors were able to succeed in formulating oil-in-water emulsions, but depending on variations in the amount of squalane and surfactants, in some cases (category B and C), some amount of excess oil was not incorporated into the emulsion.

The formulations were also tested for stability as shown below.

### Stability

Overall, the formulations of the invention showed acceptable stability under a wide range of extreme conditions of temperature, and in some cases showed remarkable stability. This is another highly beneficial technical feature of the invention. Typical emulsions, particularly oil-in-water emulsions containing such a high percentage of emollient, will inevitably separate over periods of storage, especially at higher temperatures.

As set out in Table 6 below, detailed stability testing for formulation N1 of Table 1 above was performed under incubation in containers representative of those that would be used for the final product at retail, at various temperatures (25° C., 4° C., and 45° C.) over an 18 week period. Conditions of sun exposure were also tested against a control as shown. In all cases, the formulation maintained its physical characteristics and pH within an acceptable range.

TABLE 6
Detailed Stability Results for Formulation N1

| | Day 0 | | | | | | | | |
|---|---|---|---|---|---|---|---|---|---|
| Condition | (control) | Week 1 | Week 2 | Week 4 | Week 6 | Week 10 | Week 12 | Week 16 | Week 18 |
| 25° C. | White | White | White | White | White | White | White opaque | White | White opaque |
| | opaque | opaque | opaque | opaque | opaque | opaque | Odourless | opaque | Odourless |
| | Odourless | Odourless | Odourless | Odourless | Odourless | Odourless | Water-like | Odourless | Water-like |
| | Water-like | Water-like | Water-like | Water-like | Water-like | Water-like | pH 4.51 | Water-like | pH 4.58 |
| | pH 4.55 | pH 4.55 | pH 4.52 | pH 4.54 | pH 4.54 | pH 4.58 | | pH 4.60 | |
| 4° C. | White | White | White | White | White | White | White opaque | White | White opaque |
| | opaque | opaque | opaque | opaque | opaque | opaque | Odourless | opaque | Odourless |
| | Odourless | Odourless | Odourless | Odourless | Odourless | Odourless | Water-like | Odourless | Water-like |
| | Water-like | Water-like | Water-like | Water-like | Water-like | Water-like | pH 4.51 | Water-like | pH 4.58 |
| | pH 4.55 | pH 4.56 | pH 4.51 | pH 4.55 | pH 4.53 | pH 4.54 | | pH 4.59 | |
| 45° C. | White | White | White | White | White | White | White opaque | White | White opaque |
| | opaque | opaque | opaque | opaque | opaque | opaque | Odourless | opaque | Odourless |
| | Odourless | Odourless | Odourless | Odourless | Odourless | Odourless | Water-like | Odourless | Water-like |
| | Water-like | Water-like | Water-like | Water-like | Water-like | Water-like | pH 4.54 | Water-like | pH 4.44 |
| | pH 4.55 | pH 4.51 | pH 4.51 | pH 4.53 | pH 4.55 | pH 4.57 | | pH 4.64 | |
| Sun | White | White | White | White | White | White | White opaque | White | White opaque |
| exposure | opaque | opaque | opaque | opaque | opaque | opaque | Odourless | opaque | Odourless |
| (UV; 8-10 | Odourless | Odourless | Odourless | Odourless | Odourless | Odourless | Water-like | Odourless | Water-like |
| hrs/day) | Water-like | Water-like | Water-like | Water-like | Water-like | Water-like | pH 4.57 | Water-like | pH 4.61 |
| (25° C.) | pH 4.55 | pH 4.53 | pH 4.50 | pH 4.53 | pH 4.54 | pH 4.52 | | pH 4.85 | |
| UV | White | White | White | White | White | White | White opaque | White | White opaque |
| control | opaque | opaque | opaque | opaque | opaque | opaque | Odourless | opaque | Odourless |
| (covered | Odourless | Odourless | Odourless | Odourless | Odourless | Odourless | Water-like | Odourless | Water-like |
| in foil, | Water-like | Water-like | Water-like | Water-like | Water-like | Water-like | pH 4.51 | Water-like | pH 4.58 |
| 25° C.) | pH 4.55 | pH 4.55 | pH 4.54 | pH 4.56 | pH 4.55 | pH 4.58 | | pH 4.60 | |

As shown, formulation N1 maintained its characteristics, consistency, and pH despite being subjected to extreme conditions of temperature and UV exposure. Notably, the emulsion did not separate into different layers as is typical of emulsions over time. In particular, the conditions of UV exposure and high temperature at 45° C. are likely to cause most commercial emulsions to separate into component layers. It is therefore surprising that the invented formulation remained stable under these conditions for such long periods.

Maintenance of stability over the above conditions of incubation at various temperatures and UV exposure indicates that the product has excellent shelf life. For instance, achieving 8 weeks of stability at 45° C. indicates shelf life of one year, 10 weeks of stability at 45° C. indicates that the product has a shelf life of 1.5 years, and achieving 18 weeks of stability at 45° C. indicates a shelf life of three years. Although one year of stability is not considered commercially viable, 1.5 years of stability would be acceptable and reasonable. Three years of stability is considered exceptional for a low viscosity oil-in-water emulsion.

The above results therefore reveal that this formulation has a long shelf life as is important for withstanding storage under conditions of shipping and retail.

Two further stability tests were done with similar results. Freeze-thaw testing is intended to confirm whether the product can withstand conditions which may be encountered during product shipment and warehousing. In the field, a highly stable product would be one that is able to tolerate at least two freeze thaw cycles.

The N1 formulation, as well as other formulations described herein, were put through five consecutive freeze-thaw cycles, in which samples were frozen at −25° C. for 24 hours, thawed at room temperature, and the cycle repeated four more times. At the conclusion of these freeze-thaw cycles, the sample was observed to remain white, opaque, and with a water-like consistency, and with a pH in the range of 4.50-4.55. The freeze-thaw testing is a rigorous test for personal care emulsions, and many oil-in-water emulsions would be expected to separate in the course of this testing after a single freeze-thaw cycle. The fact that N1 in particular survived five freeze-thaw cycles is remarkable.

Similarly, a further test known in the industry as “crash stability” was performed on the formulations. Samples were frozen at −25° C. for 24 hours, then incubated in a 45° C. oven for 24 hours. This cycle was repeated three times, and the formulation evaluated after each cycle. Even after three cycles, the N1 sample still remained white, opaque, with a water-like consistency, and a pH in the range of 4.50-4.55. Crash stability is considered an even more rigorous test for personal care emulsions than the freeze-thaw testing, and most oil-in-water emulsions would be expected to separate after the first round. However, surprisingly, formulation N1 as well as several others maintained their characteristics and consistency through at least two crash stability cycles. This is an exceptional stability result as well. Surviving one round of crash stability is considered good, and surviving two rounds would indicate a highly stable product. The fact that N1 survived three rounds of crash stability indicates an extremely stable product.

Stability tests were also performed on the ten further formulations described in Table 5 above. The ingredients for which amounts were kept constant between the formulations (NMF-containing ingredients, preservative, and pH adjustor) have been shown in aggregate. Indications of “stable” mean that the formulation maintained its characteristics and consistency—i.e. white opaque, odourless, water-like viscosity, and pH in the range of 4.50-4.60. The formulations are characterized below into two groups (I—high stability; and II—not sufficiently stable). The results are shown below.

TABLE 7
Stability Summaries

| | Group I | | | | | | | Group II | | |
|---|---|---|---|---|---|---|---|---|---|---|
| Formula | N1 | N2 | N3 | N4 | N5 | N6 | N7 | N8 | N9 | N10 |
| Aqueous NMF- | 6.53 | 6.53 | 6.53 | 6.53 | 6.53 | 6.53 | 6.53 | 6.53 | 6.53 | 6.53 |
| containing | | | | | | | | | | |
| ingredients | | | | | | | | | | |
| NEOSSANCE | 15.00 | 15.00 | 15.00 | 15.00 | 15.00 | 20.00 | 20.00 | 10.00 | 30.00 | 5.00 |
| SQUALANE | | | | | | | | | | |
| D.I. WATER | q.s. | q.s. | q.s. | q.s. | q.s. | q.s. | q.s. | q.s. | q.s. | q.s. |
| BRIJ IC20-70 | 1.00 | 0.50 | 1.00 | 1.50 | 1.00 | 1.00 | 1.50 | 1.00 | 1.00 | 2.00 |
| FLUIDIFEEL | 2.00 | 2.00 | 1.00 | 2.00 | 3.00 | 2.00 | 3.00 | 2.00 | 2.00 | 1.00 |
| EASY | | | | | | | | | | |
| Preservative and pH | 1.075 | 1.075 | 1.075 | 1.075 | 1.075 | 1.075 | 1.075 | 1.075 | 1.075 | 1.075 |
| adjustor | | | | | | | | | | |
| Ratio of | 5:1 | 6:1 | 7.5:1 | 4.3:1 | 3.8:1 | 6.7:1 | 4.4:1 | 3.3:1 | 10:1 | 1.7:1 |
| squalane:surfactants | | | | | | | | | | |
| Stability - | 18 | 18 | 18 | 18 | 18 | 18 | 18 | 10 | 18 | 18 |
| Incubation at 4 C. | weeks | weeks | weeks | weeks | weeks | weeks | weeks | weeks | weeks | weeks |
| Stability - | 18 | 18 | 18 | 18 | 18 | 18 | 18 | 10 | 18 | 18 |
| Incubation at 25 C. | weeks | weeks | weeks | weeks | weeks | weeks | weeks | weeks | weeks | weeks |
| Stability - | 18 | 18 | 18 | 18 | 18 | 18 | 18 | 10 | 18 | 18 |
| Incubation at 45 C. | weeks | weeks | weeks | weeks | weeks | weeks | weeks | weeks | weeks | weeks |
| Freeze-thaw cycles | Stable | Stable | Stable | Stable | Stable | Stable | Stable | Stable | Stable | Stable |
| | through 5 | through 3 | through 3 | through 2 | through 2 | through 2 | through 2 | through 3 | through 1 | through 1 |
| | cycles | cycles | cycles | cycles | cycles | cycles | cycles | cycles | cycle | cycle |
| Crash cycles | Stable | Stable | Stable | Stable | Stable | Stable | Stable | Stable | Stable | Not stable |
| | through 3 | through 2 | through 2 | through 2 | through 2 | through 2 | through 2 | through 2 | through 2 | through |
| | cycles | cycles | cycles | cycles | cycles | cycles | cycles | cycles | cycles | first cycle |

The formulations above in Group I showed good stability results, with formulation N1 showing the strongest results. While the other formulations N2-N7 were equally stable at the incubation temperatures indicated, they did not survive as many freeze-thaw or crash stability cycles. N8-N10 either did not show excellent stability when incubated, or did not pass sufficient freeze-thaw or crash cycles to be considered highly stable.

### SUMMARY

The invention has therefore been confirmed to provide a highly stable oil-in-water emulsion that has a low viscosity. It is suitable and beneficial for application to all skin but is particularly well-adapted to be applied to the scalp.

The methods described herein, particularly the method for using a microfluidizer to reduce the particle size of the formulation to turn it into a thin emulsion, are novel and result in a highly stable formulation that maintains its consistency over an extended period of storage.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An oil-in-water emulsion for topical use having a viscosity of 4.5 to 8 centiPoise, which comprises by weight, based on total weight of the emulsion, one or more oils in the total amount of 15-20% by weight, and one or more surfactants, the ratio of total oils:total surfactants being in the range of 3.8-7.5:1.

2. (canceled)

3. (canceled)

4. (canceled)

5. The oil-in-water emulsion of claim 1, further comprising 1-20% by weight of natural moisturizing factors.

6. (canceled)

7. (canceled)

8. The oil-in-water emulsion of claim 1, having a specific gravity of 0.95-1.05.

9. The oil-in-water emulsion of claim 1, having oil phase particles with a DV50 of 100-300 nm.

10. (canceled)

11. (canceled)

12. (canceled)

13. The oil-in-water emulsion of claim 1, which is stable for at least 18 weeks at 25° C.

14. The oil-in-water emulsion of claim 1, which is stable for at least 18 weeks at 4° C.

15. The oil-in-water emulsion of claim 1, which is stable for at least 18 weeks at 45° C.

16. The oil-in-water emulsion of claim 1, which is stable through at least 18 weeks of exposure to sunlight for 8-10 hours a day while incubated at 25° C.

17. The oil-in-water emulsion of claim 1, which is stable for at least two cycles of freezing at −25° C. and thawing at 45° C.

18. The oil-in-water emulsion of claim 1, which is stable after five cycles of freezing at −25° C. and thawing at 45° C.

19. The oil-in-water emulsion of claim 1 which, when applied to skin daily for two weeks, results in a 15-20% decrease in Trans-Epidermal Water Loss.

20. The oil-in-water emulsion of claim 1 which, when applied to skin daily for four weeks, results in a 20-25% decrease in Trans-Epidermal Water Loss.

21. A method for manufacturing the oil-in-water emulsion of claim 1, comprising:
(a) preparation of a first mixture of water-soluble components;
(b) preparation of a second mixture of oil-soluble components;
(c) incorporation of the second mixture into the first mixture to make an oil-in-water emulsion; and
(d) reducing the size of particles of the oil phase by using one or more of the processes of wet milling, sonication, or microemulsion.

22. The method of claim 21, in which said process is microemulsion.

23. The method of claim 22, in which said microemulsion is performed under pressure at 10,000-30,000 psi.

24. Use of the method of claim 21 to formulate an oil-in-water emulsion for topical use having a viscosity of 4.5 to 8 centiPoise, which comprises by weight, based on total weight of the emulsion, one or more oils in the total amount of 15-20% by weight, and one or more surfactants, the ratio of total oils:total surfactants being in the range of 3.8-7.5:1.

25. (canceled)

26. A leave-on product comprising the oil-in-water emulsion of claim 1 wherein the product is a leave-on composition to be applied to keratinous tissue on the scalp and allowed to remain on the keratinous tissue on the scalp.

27. A process for a nontherapeutic treatment of keratinous tissue on the scalp comprising application of the oil-in-water emulsion of claim 1 to said scalp.

28. A method of stabilizing the oil-in-water emulsion of claim 1, comprising processing of said oil-in-water emulsion through one or more of a wet mill, sonicator, or microfluidizer.

29. (canceled)
